(19) United States
(12) Patent Application Publication
(10) Pub. No.: US 2010/0125266 A1
(43) Pub. Date: May 20, 2010

(54) METHODS AND DEVICES TO TREAT COMPRESSIVE NEUROPATHY AND OTHER DISEASES

(75) Inventors: Mark Deem, Mountain View, CA (US); Vivek Shenoy, Redwood City, CA (US); Michael Hendricksen, Redwood City, CA (US); Hanson S. Gifford, III, Woodside, CA (US); Doug Sutton, Pacifica, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Foundry, LLC, Menlo Park, CA (US)

(21) Appl. No.: 12/620,304

(22) Filed: Nov. 17, 2009

Related U.S. Application Data
(60) Provisional application No. 61/148,801, filed on Jan. 30, 2009.
(60) Provisional application No. 61/115,470, filed on Nov. 17, 2008.

(51) Int. Cl. A61B 18/02; A61M 29/00; A61B 17/32

(52) U.S. Cl. 606/21; 606/192; 606/170

(57) ABSTRACT

Methods, systems and devices for treatment of musculoskeletal tissue may include one or more of stretching, scoring, cutting, and cryogenic cooling of the tissue. Exemplary usage includes the treatment of compressive neuropathy such as in carpal tunnel syndrome and plantar fasciitis. Other musculoskeletal tissues such as those in the hip, shoulder, and other regions of the body may also be treated.

### CROSS-REFERENCES TO RELATED APPLICATIONS

The present application is a non-provisional of, and claims the benefit of priority of U.S. Provisional Patent Application Nos. 61/115,470 (Attorney Docket No. 020979-004000US) filed Nov. 17, 2008; and 61/148,801 (Attorney Docket No. 020979-004100US) filed Jan. 30, 2009. The entire contents of each of the above listed applications is incorporated herein by reference.

### BACKGROUND OF THE INVENTION

1. Field of the Invention

The present disclosure generally relates to devices and methods for treating diseased or injured musculoskeletal tissues, preferably using minimally invasive techniques. Exemplary methods include dilation of tissue and/or the use of cryogenic techniques. These methods and devices may be used, for example, to treat nerve entrapment or other tissue related diseases.

Range of motion of various joints in the body may be severely limited as a result of inflammation, fibrosis or contracture of the surrounding soft tissue (e.g., tendons and ligaments) as well as adhesions between the adjacent tissues (e.g., between bone and the surrounding capsular ligaments).

Plantar fasciitis is an inflammation of the plantar fascia, a thick ligamentous/fibrous band on the bottom of the foot that is attached to the heel, and runs forward to insert into the ball of the foot. The contraction of the ligament due to inflammation and scarring can lead to significant pain when it is stretched during walking and running.

Carpal tunnel syndrome (CTS) is a common source of hand numbness and pain and results when the tendons in the wrist swell and put pressure on the median nerve, one of three major nerves responsible for supplying feeling in the hand. Recent research has shown that the incidence of CTS may be as high as 3.7% in the general population.

CTS is usually divided into three categories including, mild, moderate, and severe. Mild CTS includes sensory abnormalities alone on electrophysiologic (EDX) testing. Moderate CTS includes sensory abnormalities as well as motor abnormalities. Severe CTS includes any evidence of axonal loss (e.g., decreased or absent sensory or motor responses distal to the carpal tunnel.

Most individuals with mild-to-moderate CTS (according to EDX data) respond to conservative management, usually consisting of splinting the wrist at night time for a minimum of 3 weeks. Local steroid injection or splinting is suggested when treating patients with carpal tunnel syndrome, before considering surgery.

Patients whose condition does not improve following conservative treatment and patients who initially are in the severe CTS category (as defined by EDX) should be considered for surgery. Surgical treatment for carpal tunnel syndrome involves complete division of the transverse carpal ligament. The surgical approach may be open or endoscopic. The most commonly seen serious complications are incomplete transection of the transverse carpal ligament and inadvertent nerve or vessel injuries. Pain due to scarring at the surgical site and loss of grip strength due to resection of the ligament are also common.

An alternate procedure to ease carpal tunnel pain without cutting the carpal ligament uses a percutaneous balloon. The procedure involves inserting a balloon into the tunnel and inflating it to dilate the transverse carpal ligament, thereby increasing the spatial diameter of the carpal tunnel and relieving the pressure on the median nerve. While stretching the transverse carpal ligament may provide acute relief from the carpal tunnel syndrome symptoms, the long term efficacy of the procedure is limited since the ligament, due to its elastic nature, is likely to revert to its original dimensions.

Treatment of musculoskeletal tissues by cutting, shaving, debriding, tearing, or stretching can lead to an inflammatory response causing scarring in the treated tissue. Post-surgical pain can also be a major factor in active and passive manipulation of the treated tissue during physical rehabilitation, thereby hampering healing and slowing the return to normal function. What are needed therefore are devices and methods for treating musculoskeletal tissues which reduce the inflammation and pain associated with current surgical techniques. The methods and devices disclosed in the present invention are intended to substantially maintain the deformation of soft tissue structures like ligaments and tendons, and thereby provide extended relief from compressive neuropathies or other musculoskeletal tissue diseases and injuries.

2. Description of Background Art

Patents of interest include U.S. Pat. Nos. Re 35,523; 7,081,112; and 4,271,839.

### BRIEF SUMMARY OF THE INVENTION

Various systems, devices, and methods for treatment of soft tissue and joint diseases are disclosed herein. Exemplary use includes, but is not limited to treatment of nerve entrapment by deformation of surrounding soft tissue structures like ligaments and tendons, and thereby providing extended relief from compressive neuropathies. Other exemplary use includes treatment of tissue in the shoulder, hip, wrist, elbow, and ankle.

In a first aspect of the present invention, a method for treating compressive neuropathy in a patient comprises positioning a device with an expandable member into a space surrounding a nerve, and expanding the expandable member. Stretching soft tissue surrounding the nerve with the expandable member widens the space surrounding the nerve thereby relieving pressure exerted by the soft tissue against the nerve.

The space may comprise a carpal tunnel in a hand of the patient, and the tunnel may have a transverse carpal ligament extending transversely across the tunnel and a median nerve extending through the tunnel. Expanding the expandable member may expand it against the transverse carpal ligament, and the stretching step may stretch the transverse carpal ligament. The widening step may relieve pressure exerted by the transverse carpal ligament against the median nerve.

The device may comprise a shield that shields the nerve from pressure exerted by the expandable member. The method may further comprise scoring at least a portion of the soft tissue with a scoring element. The score may be a partial thickness score through the tissue or the score may extend all the way through the tissue. The method may also comprise cutting or cauterizing at least a portion of the soft tissue with an electrode. This may be performed by delivering radiofrequency energy to the soft tissue with an electrode coupled with the device. The cut may be a partial thickness cut or it may extend all the way through the tissue. The method may also comprise cryogenically cooling at least a portion of the soft tissue.

In another aspect of the present invention, a method for treating musculoskeletal tissue in a patient comprises positioning a device having a working surface and a distal element adjacent the musculoskeletal tissue, and engaging the musculoskeletal tissue with the distal element. The tissue is stretched into a stretched condition using the distal element, and the working surface of is cooled. This chills the stretched tissue to a cryogenic temperature so that the stretched tissue remains in the stretched condition following removal of the device.

The positioning step may comprise positioning the device into a carpal tunnel of a hand, and the tissue may comprise a ligament such as the transverse carpal ligament. The tissue may comprise plantar fascia or tissue adjacent thereto. The tissue may be disposed in a joint. The distal element may comprise an expandable member and the stretching step may comprise expanding the expandable member against the tissue. The expandable member may comprise a balloon, which may be expanded by inflation with a fluid. The stretching may comprise expanding the balloon in a first direction while constraining the balloon from expanding in a second direction. The expandable member may comprise two or more prongs and the stretching may comprise pressing the prongs against the tissue. The prongs may also be engaged against the tissue and twisted in order to stretch the tissue. The distal element may comprise a plurality of cooling tubes axially oriented and forming a generally cylindrical arrangement. The stretching step may comprise compressing the cooling tubes so as to radially deflect at least a portion of the generally cylindrical arrangement outward against the tissue.

The working surface may comprise an outer surface of a balloon, and the cooling may comprise inflating the balloon with a cryogenic fluid thereby reducing temperature of the working surface. The cooling may comprise inflating the balloon with an inflation fluid delivered through a first lumen in the device, and cooling the working surface of the balloon with the cryogenic fluid delivered through a second lumen in the device. The balloon may comprise a first chamber in communication with the first lumen and a second chamber in communication with the second lumen. The working surface may comprise an outer surface of one or more cooling tubes adjacent the distal element, and the cooling step may comprise passing a cryogenic fluid through the one or more cooling tubes. The cooling may comprise insulating at least a portion of the tissue from the cooling with an insulated portion of the device adjacent the working surface.

The method may further comprise cutting or scoring the tissue with a cutting or scoring element prior to cooling the tissue. An electrode may also be used to deliver electrical energy to the tissue in order to cut or score the tissue. The cut or score may be a partial thickness cut or score through the tissue, or it may extend all the way through the tissue. The method may also comprise cauterizing the tissue with an electrode that delivers electrical energy to the tissue. The method may comprise viewing the musculoskeletal tissue through an arthroscope.

In another aspect of the present invention, a system for treating compressive neuropathy in a patient comprises an elongate shaft having a proximal end, a distal end, and a lumen extending therebetween. An expandable member is adjacent the distal end of the shaft and has an expanded configuration and a collapsed configuration. The expandable member is fluidly coupled with the lumen, and is also positionable into a space surrounding a nerve. In the expanded configuration the expandable member is configured to stretch tissue surrounding the nerve. The system also includes a cryogenic fluid supplied through the lumen to the expandable member. The cryogenic fluid expands the expandable member into the expanded configuration, and cools an outer surface of the expandable member thereby cooling the stretched tissue.

The system may further comprise a shield adjacent the expandable member. The shield may be adapted to protect adjacent tissue from pressure exerted by the expandable member in the expanded configuration. The system may also have a scoring element disposed adjacent the expandable member, and adapted to score the stretched tissue. An electrode may be disposed adjacent the expandable member that is adapted to cut or score the stretched tissue with electrical energy. The electrode may be actuatable into an expanded configuration extending radially outward from the expandable member. The expandable member may comprise a balloon.

In another aspect of the present invention, a system for treating musculoskeletal tissue in a patient comprises an elongate shaft having a proximal end, a distal end, and a lumen extending therebetween. An expandable member is adjacent the distal end of the shaft and has an expanded configuration and a collapsed configuration. The expandable member is positionable into apposition with the musculoskeletal tissue such that in the expanded configuration, the expandable member stretches the tissue. A cooling element is adjacent the expandable member, and in fluid communication with the lumen, and a cryogenic fluid is supplied through the lumen to the cooling element. This cryogenically cools the cooling element, and thereby cools the stretched tissue.

The expandable member may comprise a first balloon and the cooling element may comprise a second balloon. The first and second balloons may be concentrically disposed about the shaft. The first and second balloons may be expandable independently of one another. The system may further comprise an inner shaft having an inner shaft lumen and a port in the inner shaft in fluid communication therewith. The inner shaft may be disposed in the elongate shaft lumen thereby forming an annular space therebetween, and the cryogenic fluid may circulate into and out of the cooling element via the annular space and the inner shaft port. The cooling element may comprise a cooling tube disposed against the expandable member that is formed into a plurality of peaks and valleys. The cooling tube may be configured to expand with the expandable member, and it may be coupled to the expandable member with a thread-like member. The cooling element may comprise a plurality of cooling tubes interwoven to form a mesh that is disposed over the expandable member.

The system may further comprise a supporting element disposed adjacent the expandable member. The supporting element may prevent expansion of the expandable member in a first direction while allowing expansion in a second direction laterally away from the supporting element. This prevents the expandable member from expanding into tissue in the first direction. The system may also comprise an insulating member disposed adjacent the cooling element. The insulating member may be configured to protect tissue from being cooled by the cooling element. The system may comprise an outer shaft having a central channel and a port in a sidewall of the outer shaft. The expandable member may be disposed in the central channel and may be configured to expand in a first direction through the port while being constrained from expansion in a second direction by an opposite sidewall of the outer shaft. The expandable member may have a plurality of cooling tubes extending axially along the elongate shaft, and the cooling element may comprise an outer surface of the cooling tubes. The cryogenic fluid may flow through the cooling tubes and axial compression of the cooling tubes may expand the tubes radially outward into a cage-like structure. The expandable member may comprise a plurality of actuatable prongs which are configured to capture the tissue therebetween. Actuation of the prongs may stretch the tissue. The system may further comprise a scoring element disposed adjacent the expandable member for scoring the tissue. The system may also comprise an electrode configured to deliver electrical energy to the tissue thereby cutting or scoring the tissue.

These and other embodiments are described in further detail in the following description related to the appended drawing figures.

### BRIEF DESCRIPTION OF THE DRAWINGS

FIG. 1 illustrates the carpal tunnel in a hand.

FIG. 2 illustrates an exemplary embodiment of a tissue dilation device.

FIGS. 3A-3B illustrate scoring of a ligament.

FIGS. 4A-4C illustrate tissue scoring and cutting devices.

FIGS. 5A-5C illustrate an exemplary embodiment of a cryogenic device.

FIGS. 6A-6C illustrate another embodiment of a cryogenic device.

FIGS. 7A-7B illustrate still another embodiment of a cryogenic device.

FIGS. 8A-8C illustrate yet another embodiment of a cryogenic device.

FIGS. 9A-9B illustrate another embodiment of a cryogenic device.

FIGS. 10A-10B illustrate expansion of a balloon in a cannula.

FIGS. 11A-11C illustrate another embodiment of a cryogenic device.

FIGS. 12A-12C illustrate still another embodiment of cryogenic device.

FIGS. 13A-13B and 14A-14B illustrate tissue stretching devices.

FIG. 15 illustrates a musculoskeletal tissue treatment kit.

### DETAILED DESCRIPTION OF THE INVENTION

Stretching and releasing soft tissue using balloon inflatable devices may be used in the treatment of compressive neuropathies. The stretching and releasing may be performed arthroscopically or with other minimally invasive techniques.

Several exemplary embodiments of devices and methods are described below which focus on the treatment of carpal tunnel syndrome. This is not intended to be limiting, and the devices and methods may be used for performing other treatments on musculoskeletal tissue, including but not limited to compressive neuropathy in the cubital tunnel, and radial tunnel, as well as other areas.

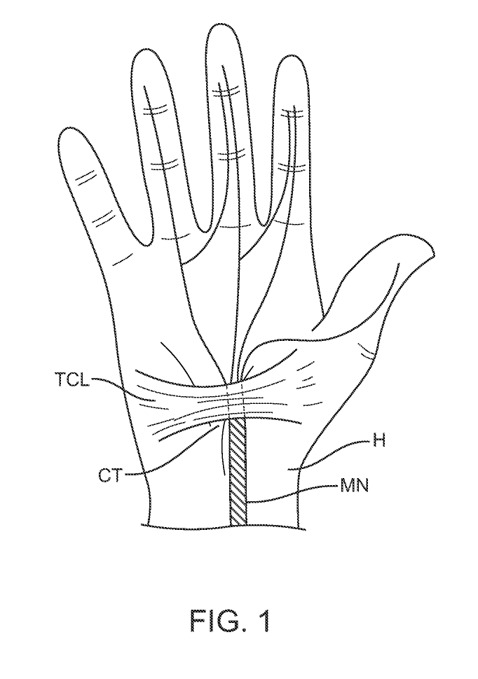

FIG. 1 illustrates basic anatomy of a hand H. The carpal tunnel CT is a narrow, tunnel-like structure in the wrist. The bottom and sides of this tunnel are formed by the carpal bones (not illustrated) and the top of the tunnel is covered by the transverse carpal ligament TCL, a strong band of connective tissue. The median nerve MN travels from the forearm into the hand through this tunnel in the wrist. The tendons that flex the fingers and thumb also travel in this tunnel.

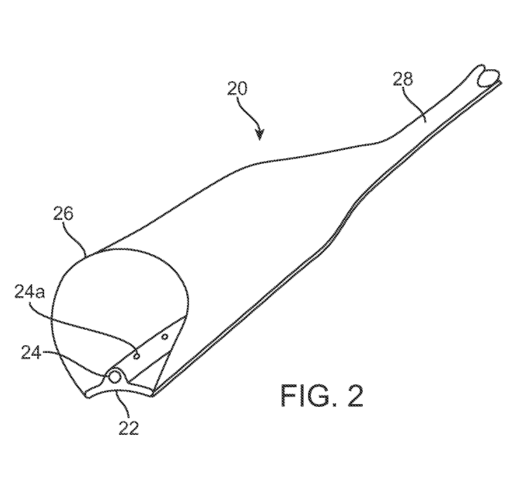

An expandable device such as a balloon or other expandable structure may be introduced into the tunnel and expanded in the tunnel space to effectively stretch the ligaments as well as release any adhesions between the capsular ligaments without disrupting the integrity of the ligament. FIG. 2 illustrates an exemplary expandable device 20 having an expandable balloon 26 coupled to a distal portion of a catheter shaft 28. The catheter shaft 28 includes an inflation lumen 24 and one or more inflation ports 24a in fluid communication with the lumen 24 for inflation the balloon 26. The balloon is substantially non-compliant and may be expanded with saline, water, contrast media, combinations thereof, or other fluids. Additionally, the inflation fluid may have a controlled temperature to warm the balloon and adjacent tissue, thereby assisting in stretching the surrounding soft tissue or releasing any adhesions. The fluid may be warmer or cooler than ambient temperature.

The device 20 is introduced into the tunnel space and optionally may have a supporting structure 22 on a side of the balloon 26 adjacent the median nerve such that, on inflation, the balloon expands primarily in the direction of the ligament so that the underlying nerve is protected from the compressive force. The supporting structure 22 which acts as a nerve protector is preferably in the form of a structure that can be introduced through a small incision in a compact form and then spreads out to cover the median nerve. Therefore, in preferred embodiments, the supporting structure 22 may be fabricated from resilient materials such as elastomers or other polymers, shape memory alloys like nitinol, or spring temper and superelastic materials such as metals. The supporting structure is delivered in a contracted configuration and then deployed into an expanded configuration.

A surface of the balloon 26 in contact with the ligament may be fabricated with a variety of coatings to minimize any friction with the balloon surface. In other embodiments, the surface may be tacky or textured to prevent any slippage between the surfaces. The balloon may be also be coated with biomaterials like hyaluronic acid or bioactive compounds like steroids. These materials may also be infused through lumens in the inflated balloon thereby forcing the therapeutic agents into the surrounding tissue.

In preferred embodiments, the physician is able to view positioning and dilation of the balloon catheter during the treatment. To provide direct visualization, an endoscope may be introduced into a lumen of the balloon catheter device 20 to evaluate the tissue pathology prior to treatment and again after the completion of the treatment. In other embodiments, the endoscope may be introduced separately from the catheter. In still other embodiments, fluoroscopic guidance may be used. To facilitate this, radiopaque markers may be placed in the balloon or along the catheter shaft to help locate the balloon in the tunnel. Furthermore, the balloon may be inflated with contrast media to enable fluoroscopic visualization during the treatment.

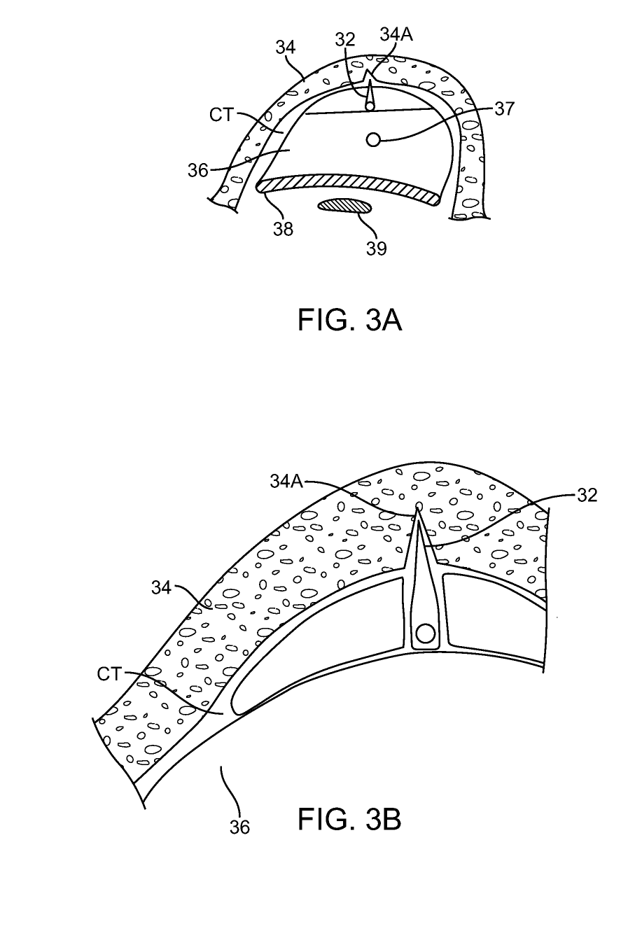

To further enable lengthening of the ligament, the surface of the balloon in contact with the ligament may have one or more scoring elements 32. FIG. 3A illustrates an end view of a balloon catheter 36 inflated in the carpel tunnel CT and FIG. 3B is an enlarged view of the scored ligament. The balloon 36 is coupled to a catheter shaft 37 and has an optional supporting structure 38 that shields the nerve 39 from compression. The supporting element generally takes the same form as the supporting element previously described in FIG. 2 above. One or more scoring elements 32 are coupled to the balloon 36 such that when the balloon is expanded, the scoring element 32 will move radially outward into the tissue, here the transverse carpal ligament 34. When the scoring element 32 pierces the ligament 34, a small incision 34a results. The incision may only partially penetrate the ligament, or the incision may extend all the way through the ligament depending on the size of the scoring element and how much the balloon is inflated.

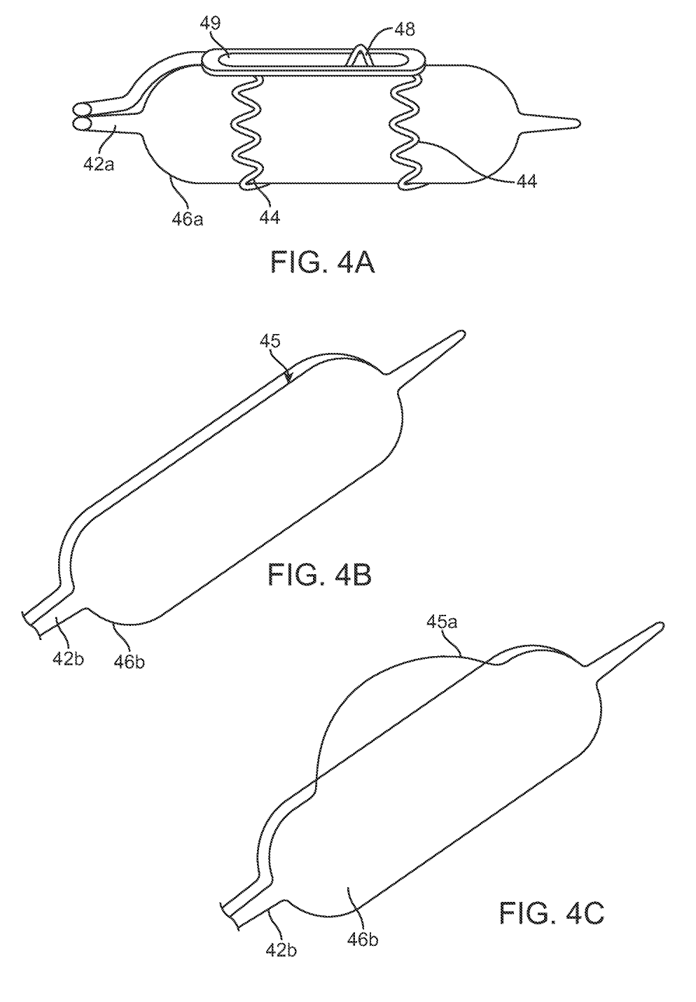

The scoring element(s) 32 may be covered at the time of introduction of the device into the tunnel space and exposed after the device is in the tunnel space. The scoring elements may be directly attached to the balloon or as seen in FIG. 4A, the scoring element 48 may be attached to a stent like structure 44 surrounding the balloon 46a that expands when the balloon is inflated. The balloon 46a is connected to a catheter shaft 42a and may have more than one stent-like structures 44 disposed on the balloon 44. The scoring elements of any of the embodiments disclosed herein may be linear, staggered, serpentine, zigzag, crossed, etc. The scoring elements may be used to create partial thickness or full thickness cuts through the ligament which is held under tension by the inflated balloon. The scoring elements may be exposed to the ligament prior to inflating to the balloon, immediately after inflating the balloon or after a period of time after the balloon has been inflated. Once the ligament has been scored, it may be placed under increasing tensile stress by inflating the balloon in a stepwise manner, thereby allowing the tissue to lengthen by plastic deformation without acutely applying excessive force.

FIG. 4A also illustrates how the scoring element may optionally also be the tip of a scoring blade that is exposed after inflating the balloon and then moved along a track 49 on the surface of the balloon, thereby scoring the inner surface of the ligament or other treatment tissue. Each device may contain one or more cutting blades/tracks.

FIGS. 4B-4C illustrate an exemplary embodiment of a balloon catheter used to dilate the tissue and also having an electrode 45. In FIG. 4B the electrode 45 is collapsed and the device may be used while the electrode remains flat, or as seen in FIG. 4C the electrode 45a may be deployed. The balloon 46b is coupled to a catheter shaft 42b and an electrode 45 runs at least partially along a surface of the balloon 46b. In the embodiment of FIG. 4B, the electrode 45 is a filament that is substantially parallel to the longitudinal axis of the balloon. One will of course appreciate that the electrode may be disposed circumferentially around the balloon, or that any number of other configurations and patterns are possible. FIG. 4C illustrates deployment of the electrode 45a radially outward away from the balloon so that it directly contacts the target tissue (e.g. ligament). In preferred embodiments, the electrode may deliver RF energy to the tissue, although other forms of energy may also be delivered with the electrode. An RF energy generator may be controlled to provide energy sufficient to create partial thickness or full thickness cuts through the ligament or other treatment tissue. The electrode may operate in a monopolar or bipolar mode.

In other embodiments, the conductive elements may also be used in conjunction with the scoring elements described earlier to enable cauterization of the scored area of the tissue, thereby preventing scarring in the area which could result in contraction of the tissue. This may be accomplished using low energy RF or other modes like cryogenic treatment of the scored areas as will be described in greater detail below.

In still other embodiments, the stretched ligament in contact with the inflated balloon may be further elongated by irradiation with a controlled laser beam or by coablation (mono or bipolar) cutting. The ligament may be cut in various patterns to enable controlled elongation using any of the techniques disclosed in this invention. The cuts may be full thickness or partial thickness cuts.

In other exemplary embodiments, the balloon may have a tapered or stepped configuration or the catheter may have multiple lumens to control the balloon inflation thereby enabling gradual and directed stretching of the ligament. The balloon may be fabricated to have more than one separate inflatable region which can be inflated independently, thereby allowing the controlled stretching of different regions of the ligament. In other embodiments, the balloon may be a linear everting balloon, thereby enabling controlled introduction and placement of the balloon in the tunnel space. An example of a linear everting balloon is disclosed in U.S. Pat. No. 4,271,839.

An inflation device such as an indeflator may be used to inflate and deflate the balloon. The inflation device may comprise a pressure sensor to enable monitoring the pressure in the balloon. The inflation device may be automated by monitoring the pressure within the balloon or the volume of fluid pumped into the balloon. The balloon may be inflated in a single step or in multiple increments with varying holding periods between each increment.

To minimize adhesions from recurring in the tunnel space, anti-adhesive materials like hyaluronic acid and other biomaterial formulations may be introduced into the joint space during or after the treatment procedure. Other bioactive materials including growth factors, corticosteroids, and anti-inflammatory compounds may be introduced into the joint space during or after the treatment procedure.

In addition to dilating and/or scoring the tissue, cryogenic devices may be used in the treatment of musculoskeletal tissue. The cooling effect helps to reduce the inflammation and pain associated with many current procedures and also facilitate tissue stretching, deformation, and other tissue manipulation. The cryogenic methods and devices disclosed herein may be used alone or in combination with other surgical devices and methods, including those for stretching, deforming, or cutting musculoskeletal tissue. The devices and methods are useful in increasing joint mobility by stretching capsular ligaments (adhesive capsulitis, etc.), relieving compressive neuropathies by stretching the surrounding soft tissue (carpal tunnel syndrome, etc.), relieving pain by stretching inflamed contracted ligaments (plantar fasciitis, etc.), and for various other purposes. Exposing diseased, stretched or otherwise injured tissue to cryogenic temperature may have a beneficial effect due to the cryo-analgesic effect in which cold temperatures provide a temporary neuropraxia or permanent denervation. Additionally, exposure to controlled cryogenic temperature has been shown to cause cell apoptosis rather than cell necrosis. Cell apoptosis in the stretched tissue minimizes any inflammatory reaction, thereby reducing the post surgical scarring and contraction of the stretched tissue.

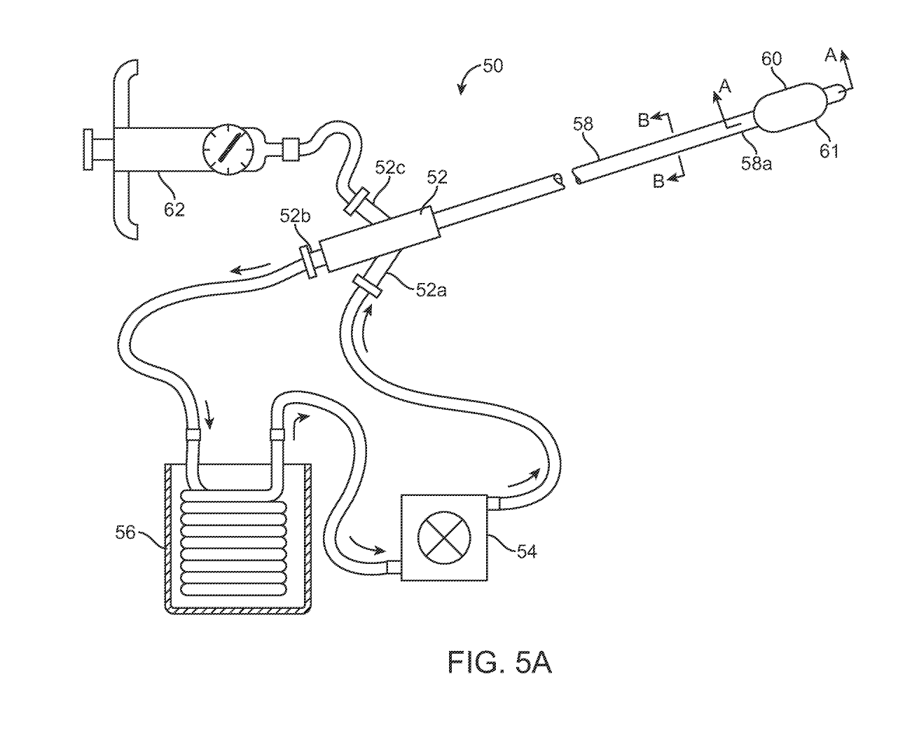

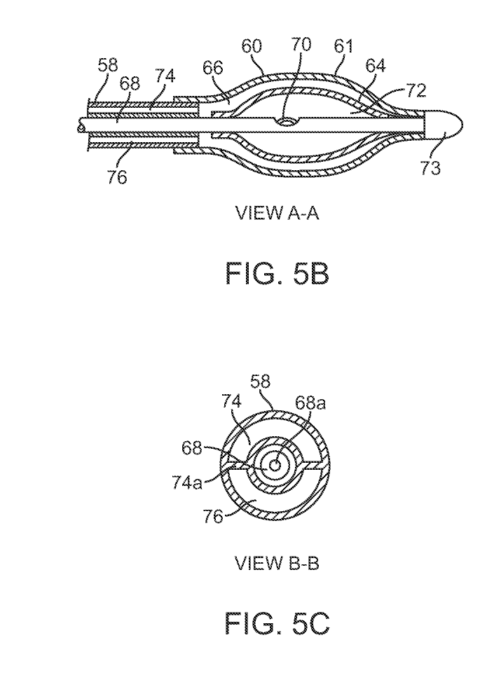

A first exemplary embodiment of a cryogenic system 50 is illustrated in FIGS. 5A-5C. FIG. 5A illustrates an overview of the system, FIG. 5B illustrates a cross-section of the working end of the device, and FIG. 5C illustrates a cross-section of the shaft. The cryogenic system 50 includes an elongated flexible shaft 58 having an expandable balloon 60 with a cryogenic surface 61 near the distal end of the shaft 58. The proximal end of the shaft 58 includes an adaptor fitting 52 which allows various connections to be established with lumens and channels in the shaft. A balloon inflation/deflation device 62 is fluidly coupled with the balloon 60 via a port 52c on the adaptor 52, and cryogenic cooling fluid flows into and out of the shaft via ports 52a, 52b, respectively. The system also includes a cooler unit 56 for storing and/or cooling the cryogenic fluid and a pump 54 for circulating the fluid through the system 50.

The expandable balloon 60 is configured to radially expand from a low profile contracted configuration to a larger profile expanded configuration. It will generally be a non-compliant balloon, although compliant and semi-compliant balloons may be utilized. The expandable member 60 includes or is coupled to a cryogenic working surface 61 adapted to be chilled to cryogenic temperatures and to chill the target tissue, usually by direct contact with the tissue. FIG. 5B is a cross-sectional view of the working end of the device taken along the line A-A in FIG. 5A. The working end of the device includes an inner shaft 68 disposed in an outer shaft 58. A tapered distal tip having a rounded nose 73 facilitates introduction of the device into the target tissue and minimizes trauma. A proximal end of the outer balloon 60 having cryogenic working surface 61 is coupled to the distal end of the outer shaft 58 and the distal end of the outer balloon 60 is coupled to a distal portion of the inner shaft 68. The inner balloon 64 is disposed within the outer balloon 60 and its proximal and distal ends are coupled to the inner shaft 68. The inner shaft 68 has an inflation lumen 68a (best seen in FIG. 5C) that is in fluid communication with the inner balloon via port 70 for inflating the inner balloon 64 thereby expanding the target tissue. Port 70 may be formed by skiving the inner shaft 68 so that inflation fluid can exit the inflation lumen 68a and expand inner balloon 64. The outer shaft preferably has an input lumen 74 and an output lumen 76 for supply and return of the cryogenic fluid to the cryogenic chamber 66 formed by the space between the inner 64 and outer 60 balloons. This permits continuous circulation of cryogenic fluid through the system. The cryogenic chamber may cover the entire circumference of the balloon or only part of the circumference thereby limiting the cryogenic exposure only to the tissue of interest.

In alternative embodiments, the cryogenic medium may fill the inner balloon, and a saline solution may be used to fill the outer balloon. The introduction of cryogenic medium into the inner balloon reduces the temperature of the surrounding fluid medium. By using the appropriate inflation medium, the temperature to which the tissue is exposed may be controlled. For example, by altering the salt concentration of the saline solution in the outer balloon, the freezing point of the solution may be depressed, thereby restricting the drop in temperature to the freezing temperature of the saline solution and relying on the latent heat of freezing the saline solution to control the temperature of the outer balloon. By having the cryogenic medium in the inner balloon, the risk of releasing the cryogenic medium into the surrounding tissue is minimized. The outer balloon may also be divided into more than one segment and the inner balloon may be partially insulated in order to limit the cryogenic exposure.

FIGS. 5A-5B show an embodiment having a single cryogenic chamber, although other embodiments may have multiple cryogenic chambers which may be expanded and/or cooled independently of one another. FIG. 5C illustrates a cross-section of the outer shaft 58 taken along the line B-B in FIG. 5A and more clearly illustrates the lumen configurations of the inner 68 and outer 58 shafts. The outer shaft 58 is divided by an axial septum 74a into upper and lower lumens 74, 76, one of which may be used to deliver cryogenic fluid into the cryogenic chamber, while the other is for the return of the cryogenic fluid from the cryogenic chamber. In alternative embodiments, only a single lumen may be provided. The cryogenic working surface 61 is in thermal communication with the cryogenic chamber 66. The shaft 58, expandable member 60, and working surface 61 are preferably dimensioned to permit introduction through an access port in the skin to the target tissue, the distal portion of the apparatus typically having a cross-section with a maximum transverse dimension from 5 mm to 50 mm, more preferably from 10-40 mm, and most preferably from 15 to 35 mm.

The shaft 58, made of a biocompatible polymer or metal, may be rigid or flexible, resilient or malleable, or any combination thereof at various points along the length of the shaft, depending upon the procedure in which it is to be used and the location of the target tissue. The shaft 58 may also be shapable, deflectable or steerable by means of any of various known catheter steering mechanisms. The shaft has a working length selected to reach the target tissue while allowing effective manipulation of the apparatus from outside the body, preferably being from about 10 mm to 100 mm, more preferably from about 20 mm to 70 mm, and even more preferably from about 30 mm to 50 mm.

In its expanded configuration, the expandable member 60 may have various shapes depending upon the procedure to be performed and the shape and location of the target structure to be treated. In exemplary embodiments, the expandable member may be totally or partially spherical, ellipsoid, conical, kidney shaped, cylindrical or sausage shaped, disk-shaped, racetrack-shaped, or shaped in any of various other symmetrical or asymmetrical forms. In the contracted configuration the expandable member will be of minimal cross-sectional size, preferably being no more than about 5 mm, more preferably less than about 4 mm, in it largest transverse dimension, such that it may be inserted through a tubular access port suitable for arthroscopic or other endoscopic procedures.

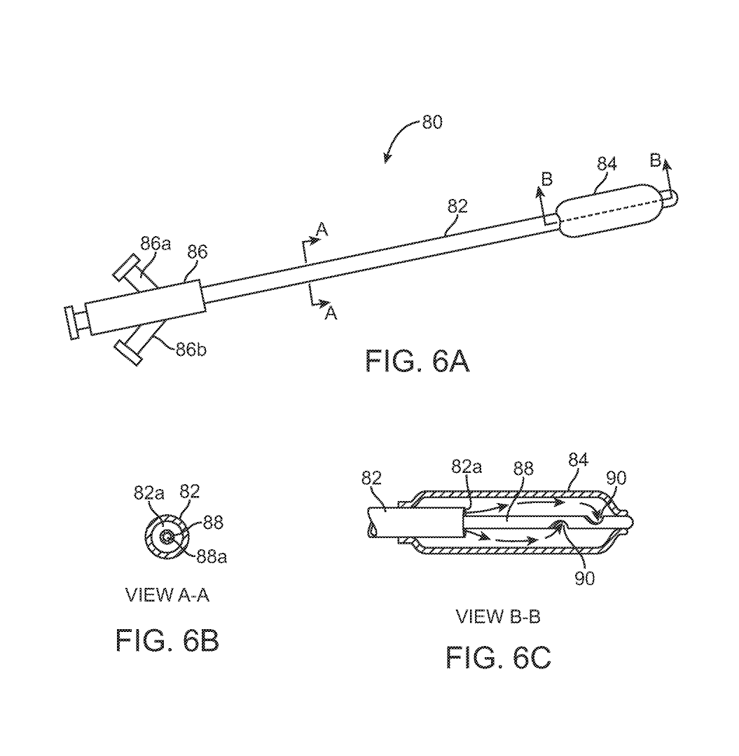

FIGS. 6A-6C illustrate an exemplary embodiment of a cryogenic system having a single-walled, single chamber balloon. Cryogenic fluid is used to inflate the balloon. FIG. 6A illustrates an overview of the system, FIG. 6B is a cross-sectional view taken alone line A-A, and FIG. 6C is a cross-section taken along line B-B. The system 80 includes an elongate flexible shaft 82 with an expandable balloon 84 near the distal end of the shaft and a fluid adapter 86 near the proximal end of the shaft. An inner shaft 88 is disposed in the outer shaft 82 (best seen in FIG. 6B-6C). The adapter 86 may have any number of ports, however in this embodiment the adapter has two ports. One port 86a allows cryogenic fluid to be introduced into the balloon 84 via a lumen 82a formed by the annular space between the inner 88 and outer shafts 88. The other port 86b allows cryogenic fluid to exit the balloon 84 via ports 90 in inner shaft 88 and lumen 88a to return to a cooler or cryogenic fluid reservoir. One will appreciate that flow may be in the opposite direction. The proximal end of the balloon 84 is attached to a distal portion of the outer shaft 82 and the distal end of the balloon 84 is attached to a distal portion of the inner shaft 88.

In any of the balloon embodiments, the expandable member will be inflatable to sufficient pressures to perform the applicable procedure. Typical pressures are in the range of 2-20 atmospheres. The balloons may be inflated using a conventional inflation fluid medium such as saline, a gaseous fluid such as air or carbon dioxide, or a cryogenic medium. The design and construction of cryogenic balloons and catheters have been disclosed in U.S. Pat. No. 7,081,112 which is incorporated herein in its entirety.

The cryogenic medium used to cool the tissue of systems disclosed herein may be any of a variety of different cooling media. One simple approach is to circulate a cooled liquid, such as saline, through the balloon or other device. A salt solution may be used with its freezing point specifically targeted so that, due to the latent heat of fusion, it tends to maintain a specific desired temperature. The saline may be circulated through the balloon by a pump from an external cooling system. Alternatively, the balloon may be cooled by a gas such as nitrous oxide using the Joule-Thomson effect. In this approach, gas or liquid flows into the balloon at very high pressure, and within the balloon the gas expands and its pressure drops dramatically before it is vented out of the balloon, causing its temperature to drop dramatically. These two methods may also be combined, to rapidly cool the balloon to a specific temperature.

The balloon systems may be paired with a mechanical structure to facilitate two separate cryogenic treatments of the target tissues. For instance, an external expandable structure or cage may surround the balloon concentrically, or a portion of the balloon in the case where directional treatment is desired. This expandable structure may be constructed, for example, of hollow hypotubes or hollow polymer tubes designed to withstand cryogenic temperatures. These hollow tubes may be aligned longitudinally along the axis of the balloon, arranged spirally around the balloon, or arranged in rings around the circumference of the balloon.

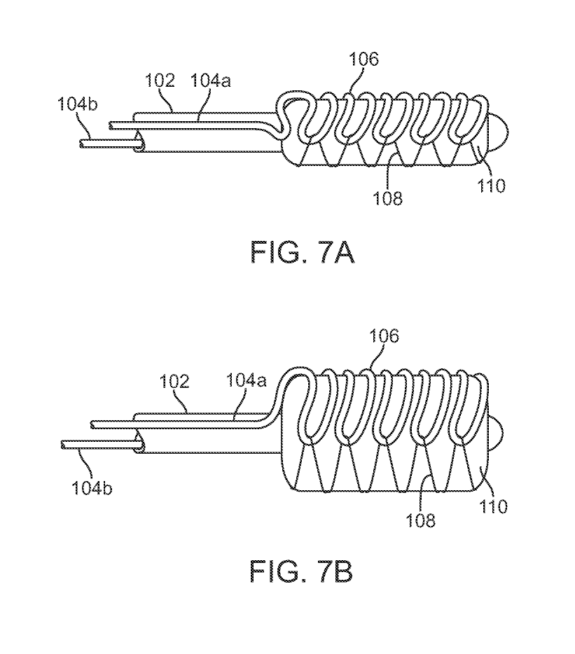

FIG. 7A shows an embodiment having an unexpanded balloon and mechanical structure. FIG. 7B shows the device in the expanded configuration. The device shaft 102 includes an expandable balloon 110 attached to the distal end and having a continuous cooling tube 106 tube bent transversely into a series of loops or waves forming peaks and valleys, which have a curvature about the longitudinal axis of the balloon 102 so as to have a cylindrical shape conforming to the shape of the balloon 102. The tube may comprise a metal hypotube or other material having suitable thermal properties for cryogenic treatment and the flexibility and resilience to be expandable as the balloon expands. Input and output tubes 104a, 104b extend axially along the device shaft 102 and allow coolant to flow from a coolant source through the cooling tube 106. The tube 106 is held on to the balloon 110 by an elastic thread 108 woven between opposing loops in the tube and adapted to elongate as the balloon expands.

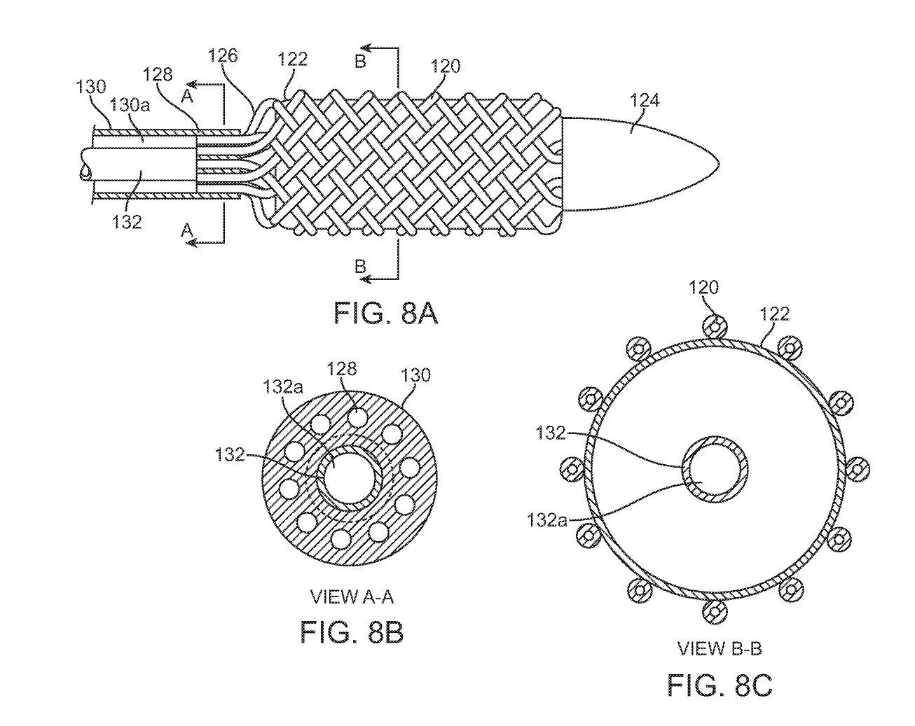

Alternatively, as shown in FIGS. 8A-8C, the expandable structure may comprise a plurality of tubes 120 woven into a cylindrical cage which surrounds the balloon 122 and is expandable with it. FIG. 8B shows a cross-section taken along line A-A and FIG. 8C shows another cross-section taken along line B-B. The tubes 120 are connected by flexible connector tubes 126 to a manifold 128 at the distal end of the outer shaft 130 of the device, which communicates with the cryogenic fluid delivery lumen 130a in the shaft 130. An inner shaft 132 has an inflation lumen 132a for inflating and deflating the balloon 122. The balloon may be inflated with water, saline, contrast media, gas, or other fluids commonly used in balloon inflation. The balloon may also be inflated with a cryogenic fluid. The device also includes a rounded and tapered distal tip 124 to facilitate delivery of the device through tissue without causing trauma. In one embodiment, the hollow tubes provide cryogenic temperatures to the tissue which are lower than the cryogenic temperatures provided by the balloon itself. For example, the balloon may be designed to provide temperatures in the −5° C. to −15° C. range to induce apoptosis to the entirety of the stretched tissue, while the hollow tubes provide colder cryogenic therapy to facilitate stretching or to provide a more profound or long lasting neuropraxia or denervation.

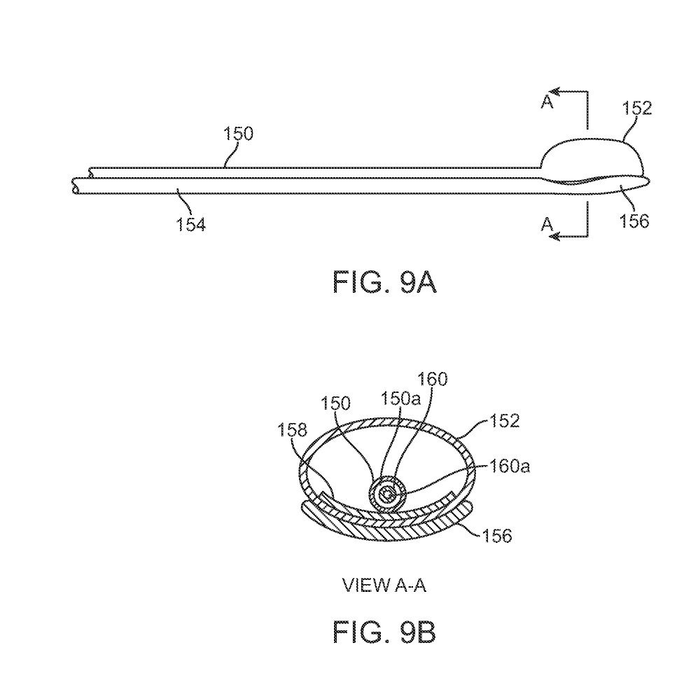

As shown in FIG. 9A, the cryogenic device may have a supporting structure or shield 156 on one side such that, on inflation, the balloon 152 expands primarily on the side in contact with the tissue to be treated. The balloon 152 is coupled to shaft 150 and an inner shaft 160 may optionally be disposed in shaft 150, forming an annular space 150a, and an inner shaft lumen 160a for inflating and deflating the device. FIG. 9B illustrates a cross-section of FIG. 9A taken along line A-A. The cryogenic device may be mounted on a lateral side of the supporting structure 156 so as to expand laterally in one direction away from the balloon shaft 150. The supporting structure 156 may be either flexible or rigid, and may have a distal end adapted to perform certain functions such as dissecting, tunneling through or retracting tissue, or the like. In the embodiment shown, the supporting structure 156 is attached to an elongated rigid shaft of titanium or stainless steel 154 and has a flattened and rounded spoon-like distal tip to facilitate positioning the expandable member 152 within a narrow tissue or joint space. Optionally, one side of the balloon 152 may have a layer of insulation 158 mounted internally or externally (best seen in FIG. 9B) on the side adjacent the supporting shaft 154 so that cryogenic temperatures are directed in a specific direction relative to the supporting structure. Optionally, a heating element (not shown) may be mounted in the tip of the supporting shaft and connected to an electrical lead extending through the shaft, allowing the tip of the shaft to be heated. Preferably the heating element is separated from the expandable member by thermal insulation. This enables the delivery of heat from the tip of the supporting shaft so that tissues other than those targeted for treatment may be maintained at temperatures above cryogenic levels, or selected tissues may be heated to higher temperatures for various therapeutic purposes.

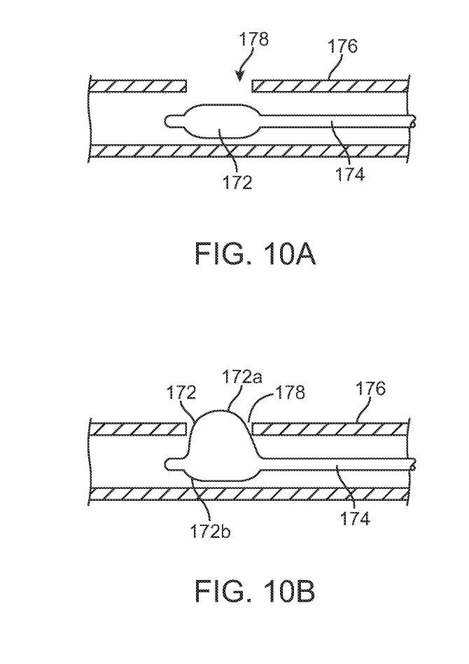

As an alternative for delivering cryogenic treatment in a specific direction, the device may be inserted through a tubular cannula having a side window along a lateral side through which the balloon may be expanded. FIG. 10A shows a balloon 172 in an unexpanded configuration attached to shaft 174. The balloon 172 is advanced through a cannula 176 until the balloon 172 is adjacent the side window 178 in cannula 176. FIG. 10B shows expansion of the balloon 172 with one side of the balloon 172b constrained by the cannula 176 and the opposite side of the balloon 172a expanding through the side window 178 such that it can engage tissue.

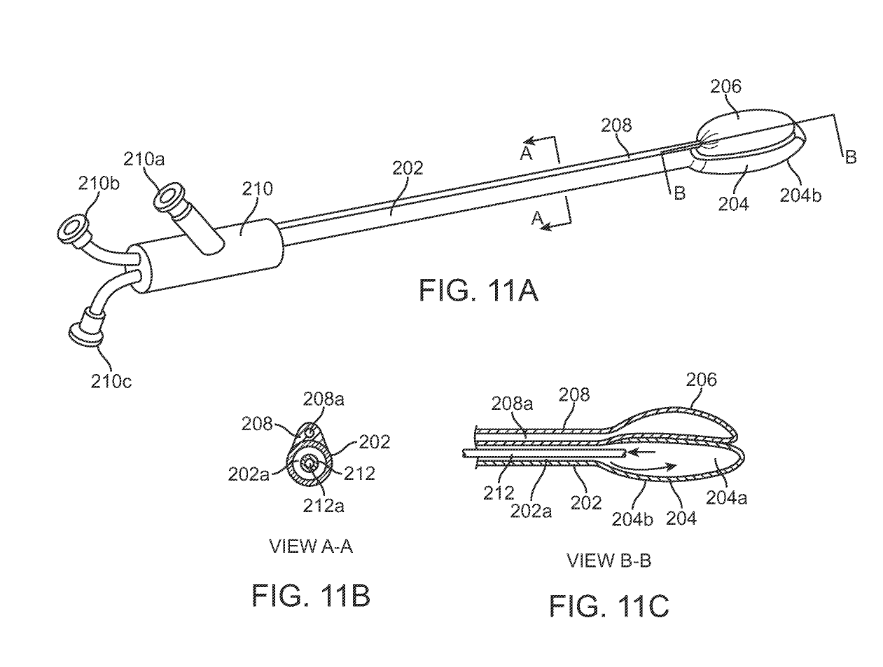

In an alternative embodiment, shown in FIGS. 11A-11C, a cryogenic apparatus comprises a balloon and a cryogenic probe. FIG. 11A illustrates an overview of the cryogenic device and FIGS. 11B-11C show cross-sections taken along lines A-A and B-B, respectively. The elongated probe has a distal tip 204 with a working surface 204b adapted to engage the target tissue and an adaptor 210 near the proximal end of the device has a plurality of connector ports 210a, 210b, 210c to allow cryogenic fluid to be delivered to and returned from the device and also to allow inflation/deflation of the balloon. The distal tip 204 has an interior chamber 204a is coupled to an elongate shaft 202 having an inner shaft 212 disposed therein thereby forming an annular space 202a between the inner shaft 212 and the elongate shaft 202. The inner shaft 212 also has a central lumen 212a. In use, cryogenic fluid may be delivered through the annular space 202a to the interior chamber 204a in distal tip 204 with return flow flowing back through lumen 212a. Optionally, as shown in FIG. 11A, the device may further include an expandable member 206, e.g. balloon, mounted on a lateral side of the probe adjacent to the distal tip 204 which may be expanded to urge the working surface 204b into engagement with the target tissue. An inflation lumen 208a formed in a shaft 208 provides a fluid pathway for inflation fluid. The shaft 208 may be integral with the cryogenic probe shaft 202 as seen in FIG. 11B, or it may be a discrete shaft. The distal tip 204, or that portion thereof containing the working surface 204b, will be constructed of a metal or other heat-conducting material such that delivery of a cryogenic fluid through the delivery lumen into the interior chamber cools the working surface to cryogenic temperatures. The working surface may extend around the circumference and/or distal end of the distal tip such that the entire tip is cooled to cryogenic temperatures, or the distal tip may be partially insulated or made of an insulating material such that the cryogenic working surface is limited to a specific portion of it, e.g. the side facing away from the balloon as shown in FIG. 11A. The shape and character of the working surface is adapted for the procedure and tissue structure to be treated, and in various embodiments may be flat, curved, spherical, concave, convex, smooth, dimpled, slotted, grooved, bumpy, sticky or slippery, or may include depressions or protrusions of various configurations. The distal end of the probe may be steerable or shapable using mechanisms known in the medical device arts. The probe may be further adapted to perform other functions, such as grasping or clamping a tissue structure in contact with the working surface during cryogenic treatment.

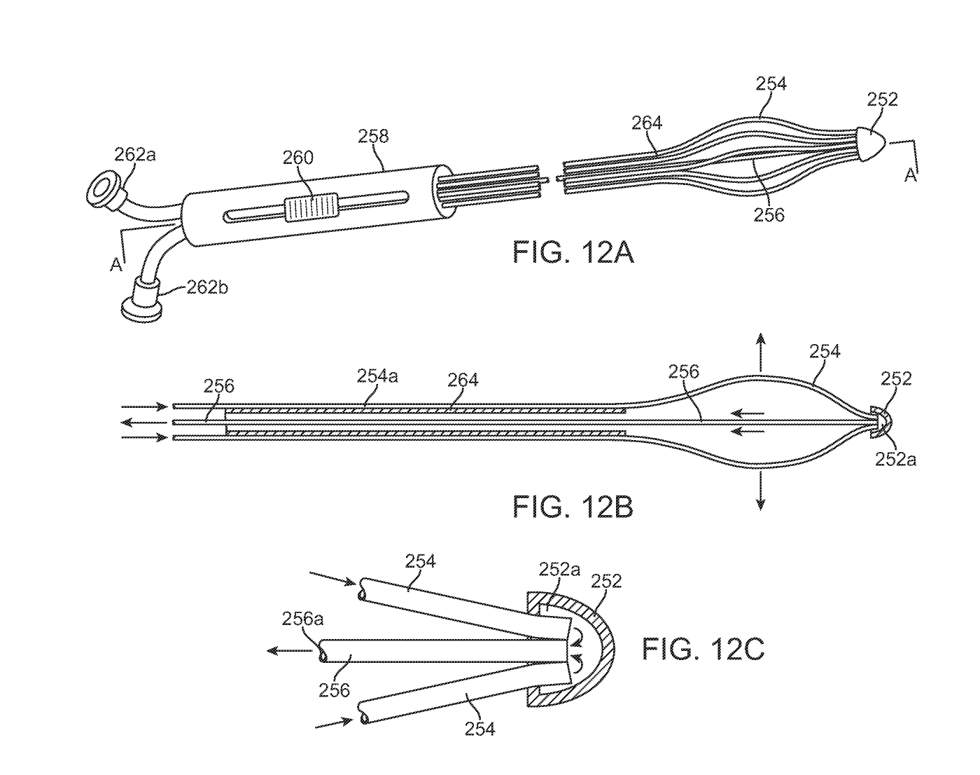

In another embodiment, the cryogenic treatment and/or tissue stretching is achieved by a purely mechanical device without the use of an adjunctive balloon to provide the expansion. FIG. 12A shows a perspective view of the device, while FIG. 12B shows a cross-section taken along line A-A, and FIG. 12C highlights a distal portion of the device. In this embodiment, a hollow cage comprising a cylindrical arrangement of longitudinally oriented tubes 254 extends proximally over an elongate shaft 264 into a manifold (not shown) in the handle 258 which has a sliding actuator mechanism 260 and inflow and outflow ports 262a, 262b for coupling with a cryogenic fluid source. The device also includes a central shaft 256 that is operably coupled with the actuator mechanism 260 such that retraction of the slider 260 will pull the distal nose cone 252 proximally, thereby forcing the tubes 254 to radially expand outward into an expanded configuration. In the expanded configuration, the tubes 254 provide the radial force necessary to deform the tissues. Advancing the actuator 260 proximally will collapse the tubes 254.

Central shaft 256 has an inner lumen 256a extending to the distal end where a manifold connects the lumen 256a with the hollow deflectable tubes 254. The central deployment shaft 256 may also be tubular and may be in communication with the distal ends of the hollow tubes 254 within the nose cap 252 as seen in FIG. 12C, to allow cryogenic medium from the deflectable members 254 to return proximally. In this embodiment, the tubes 254 provide both the stretching and cryogenic delivery functions. In alternative embodiments, the cage formed by the tubes 254 may be constructed so that stronger outer members provide the main deformation force to the target tissues, while other outer member carry the cryogenic medium. In other embodiments, deformation members may alternate with cryogenic members, or all of one type of member may be arranged on one side of the cage, or in any other desired combination in order to deliver force and cryogenic therapy directionally. The force-applying members may be thicker-walled tubes, or solid members. The cryogenic applying tubes may be thinner walled members, or members of another mechanically weaker material, such as a polymer tube designed to withstand cryogenic temperatures. In addition, one of skill in the art will appreciate that the expandable cage in FIG. 12A may also be disposed over an inflatable balloon to provide the desired expansion in place of the central shaft shown. The balloon member may be constructed of a polymer material reinforced with a fabric, polymer or metal in order to provide greater strength and/or temperature resistance for cryogenic applications. In some embodiments, these reinforcements may be a braided material. The balloons disclosed in this embodiment as well as in others described above may be in direct contact with the soft tissue to be treated (e.g. ligament, tendon, muscle, etc.), in contact with tissues covering the surfaces of such tissues (e.g. synovial tissue, fascia, etc.), or simply placed in close proximity to the tissues to be cooled so as to chill them to cryogenic temperatures. Additionally, any of the cryogenic devices disclosed herein may be combined with the scoring embodiments previously discussed above to further facilitate lengthening, stretching, or otherwise deforming the soft tissue.

In alternative embodiments, the expandable portion of the device may be made out of a woven metal braid such that foreshortening with a central member as described above provides outward stretching force. The cryogenic members may be any of the arrangements described above in the balloon or cage embodiments.

The temperatures used in cryogenic embodiments disclosed herein will generally range from −5° C. to −50° C., preferably from −5° C. to −30° C., and most preferably from −5° C. to −15° C. The temperature selected may also depend upon the nature of the treatment provided. In order to reduce inflammation and/or provide apoptosis to allow quiescent tissue recovery after stretching or injury, temperatures may be higher than those appropriate for cryogenic treatment of the tissue prior to or during stretching to facilitate the stretching itself and/or inhibit recoil.

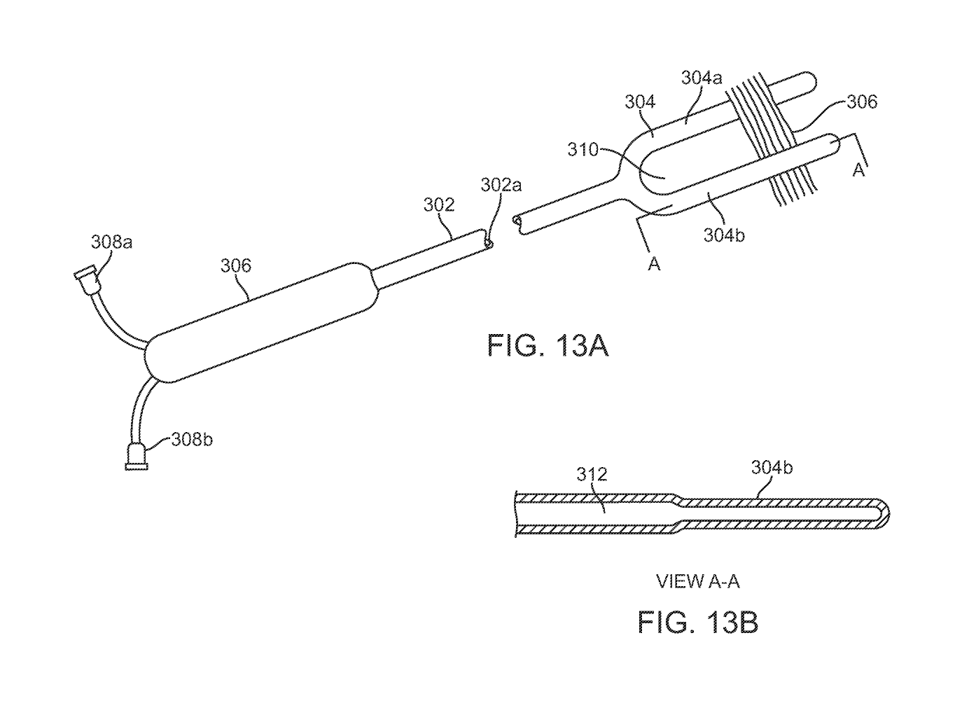

The cryogenic devices of the invention may be adapted for stretching or otherwise deforming tissue in conjunction with cryogenic cooling. FIGS. 13A-13B illustrate one exemplary embodiment having a pair of prongs. FIG. 13A illustrates an overview of the device and FIG. 13B shows a cross-section of one of the prongs taken along the line A-A. The cryogenic device includes a fork-like distal end 304 with a pair of fixed, blunt-tipped axial prongs 304a, 304b separated by a gap 310. The fork-like distal end 304 is attached to an elongate shaft 302 having a central lumen 302a which has a handle 306 at the proximal end. A pair of adapters 308a, 308b allow inflow and outflow of cryogenic cooling fluid to the prongs 304a, 304b. The prongs 304a, 304b have hollow interiors 312 (best seen in FIG. 13B) which are in communication with a fluid delivery lumen 302a in the shaft 302 to allow delivery of the cryogenic fluid. The prongs are made of a thermally conductive material so that their exterior surfaces will be cooled to cryogenic temperatures. The width of the gap between the prongs is selected to allow the prongs to be positioned around a target tissue 306 such as a ligament and to stretch the tissue by twisting around the longitudinal axis of the device.

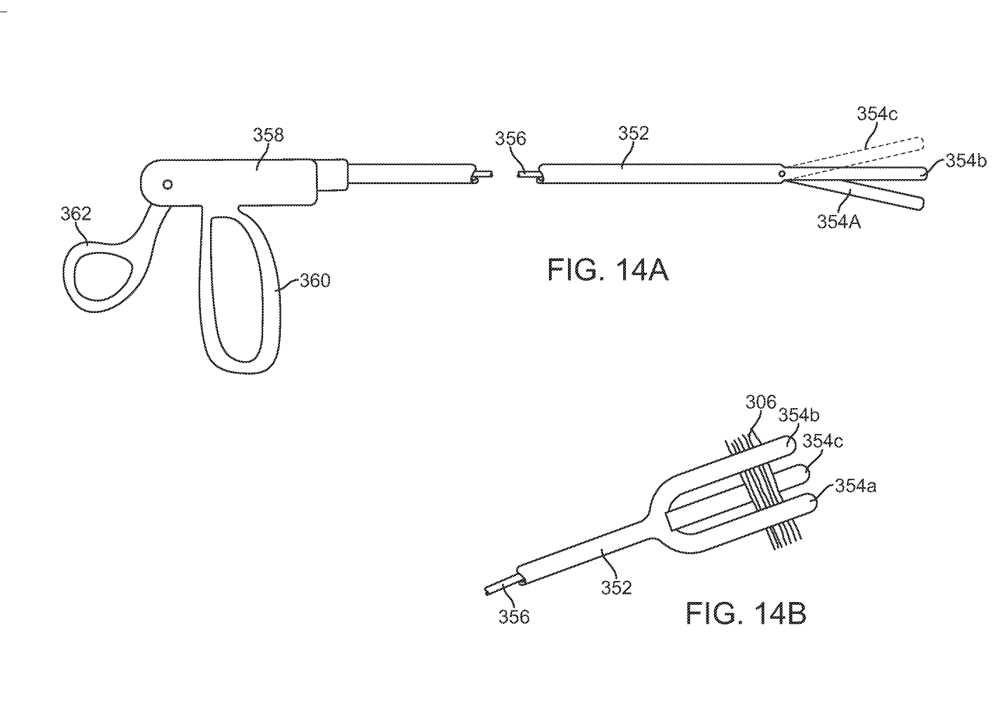

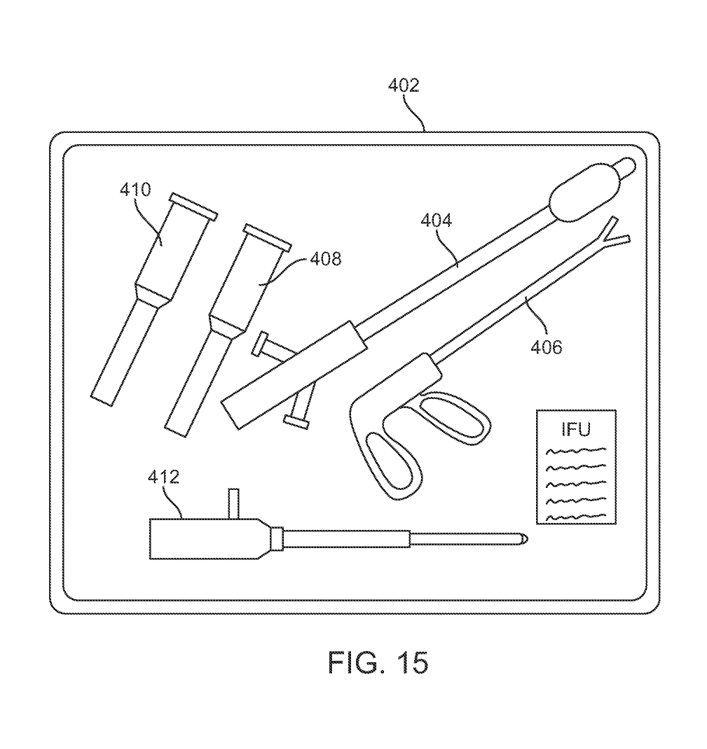

In an alternative embodiment, shown in FIGS. 14A-14B, the cryogenic device has a pair of fixed, hollow prongs 354a, 354b, and also includes a third articulating prong 354c disposed between the fixed prongs. The third prong 354c articulates about a transverse axis by means of an actuator 362 and hand grip 360 on handle 358 at the proximal end of the device which exerts tension on a wire 356 extending through the outer shaft 352 of the device and coupled to the articulating prong 354c. In this way, a piece of tissue 306 may be trapped between the lower surfaces of the fixed prongs 354a, 354b and the upper surface of the articulating prong 354c. As shown in FIG. 14B, the tissue 306 may be stretched by pivoting the articulating prong 354c upwardly between the fixed prongs 354a, 354b until the desired degree of deformation has been achieved. Cooled cryogenic fluid may be simultaneously circulated through the fixed prongs to cool the stretched tissue 306, thereby reducing the force required to stretch the tissue, along with the resiliency of the tissue so that it remains stretched, and the inflammation and pain associated with the stretching. Optionally, the articulating prong may be hollow and in communication with the fluid delivery lumen in the shaft so as to also cool the tissue along with the fixed prongs.

The invention further provides kits containing instructions for use IFU, and one or more of the cryogenic devices described herein as well as additional devices useful in performing various therapeutic procedures. As shown in FIG. 10, such kits 402 may include, in addition to one or more of the cryogenic devices 404 described above, interventional devices to be used in conjunction with the cryogenic devices of the invention such as tissue cutters 406, tissue stretchers, debridement devices, suction 408 and irrigation 410 devices, tissue retractors, ablation devices and other instruments useful in treating musculoskeletal tissues, as well as arthroscopic access ports 412, arthroscopic visualization devices and accessories, cryogenic fluid pumps, coolers, fluid containers, tubing, and related fittings and accessories.

In any of the embodiments described herein, the cryogenic devices may include a sensor and/or transmitter coupled to the device near its distal end to detect one or more conditions at the surgical site. For example, a sensor may be provided on the device to detect the position of other surgical instruments being used in the surgical procedure in order to allow the operator to target particular locations for the treatment, to avoid particular anatomical structures, or to avoid contact with an expandable member on the cryogenic device to avoid puncture or other damage. Such a sensor may be a piezoelectric sensor, infrared sensor, capacitance sensor, Doppler sensor, ultrasound sensor or other sensor suitable for detecting the proximity of other structures. The sensor may be mounted within the expandable member of the device, on the shaft of the device, or on another portion of the device near its distal end. The sensor may be coupled to a conductive wire extending to the proximal end of the device for coupling to a power source and signal processor, or the sensor may be coupled to a wireless transmitter to convey a signal via radio waves. Other sensors that may be included on the device include temperature sensors, pressure sensors, and other known sensors. Alternatively or additionally, an electronic transmitter or beacon may be provided on the device to allow the precise location of the device to be detected by an external position detector. The device may also be provided with radiopaque markers for viewing the device using fluoroscopy.

The devices of the invention may also be equipped with other features useful in surgical procedures. For example, a light source such as an incandescent, LED, or halogen bulb may be mounted near the distal end of the device, or an optical fiber may extend through the device from a light source outside the body. The device may also include an irrigation or aspiration lumen for delivering irrigation fluid to or aspirating fluids from the surgical site. A lumen suitable for introducing an arthroscope may also be provided in the device. The device may alternatively have end effectors mounted thereto which may be actuated from the proximal end, such as graspers, retractors, shavers, perforators, or cutters.

In preferred embodiments, the methods of the invention are performed arthroscopically. In such methods, the cryogenic devices will be introduced through arthroscopic access ports and under visualization using an arthroscope. The cryogenic devices of the invention are preferably adapted for such arthroscopic procedures, being configured in a low profile to facilitate introduction through small tubular ports in the skin, being readily visualized with an arthroscope, and being adapted to allow user control and manipulation from the proximal end of the device.

The devices and methods are particularly useful for the treatment of compressive neuropathies such as carpal tunnel syndrome. The cryogenic devices disclosed herein may be used to expose the carpal ligament and tendons surrounding the median nerve to cryogenic temperatures, thereby reducing inflammation and providing neuropraxia or denervation so as to reduce pain. In preferred embodiments the cryogenic devices will be adapted to expose only the ligaments, tendons and/or other tissue surrounding the median nerve to cryogenic temperatures, while protecting the median nerve itself from such exposure. For example, cryogenic devices which are adapted to expand only in one direction, or which are insulated on those surfaces which engage the median nerve, both described above, may used to shield the median nerve from cryogenic exposure.

In a preferred method of treating carpal tunnel syndrome, the cryogenic devices are inserted transcutaneously into the wrist or hand, preferably through a tubular access port, and brought into engagement with or in close proximity to the carpal ligament. Upon introduction of a cryogenic fluid into the device, the carpal ligament is cooled to cryogenic levels prior to or while the ligament is stretched, thereby reducing the force required to achieve the desired degree of stretch, and/or reducing the tendency of the ligament to recoil following stretching. In such methods, the carpal ligament may be stretched using the same cryogenic balloon or other expandable device used to deliver cryogenic therapy, or using a separate balloon or device. Alternatively or additionally, the cryogenic devices may be used to expose the transverse carpal ligament to cryogenic temperatures following stretching and/or cutting, partial cutting, or scoring of the ligament, thereby reducing inflammation and providing neuropraxia to enhance recovery. In some embodiments, the temperatures applied prior to or during stretching of the ligament may be different, typically lower, than those applied following stretching.

The devices and methods are exemplary and not limiting. Other musculoskeletal pathologies may be treated using the devices and methods disclosed herein. While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for treating compressive neuropathy in a patient, said method comprising:
positioning a device with an expandable member into a space surrounding a nerve;
expanding the expandable member;
stretching soft tissue surrounding the nerve with the expandable member; and
widening the space surrounding the nerve thereby relieving pressure exerted by the soft tissue against the nerve.

2. The method of claim 1, wherein the space comprises a carpal tunnel in a hand of the patient, the tunnel having a transverse carpal ligament extending transversely across the tunnel and a median nerve extending through the tunnel.

3. The method of claim 2, wherein the expanding expands the expandable member against the transverse carpal ligament.

4. The method of claim 3, wherein the stretching stretches the transverse carpal ligament.

5. The method of claim 2, wherein the widening relieves pressure exerted by the transverse carpal ligament against the median nerve.

6. The method of claim 1, wherein the device comprises a shield, the method further comprising shielding the nerve from pressure exerted by the expandable member with the shield.

7. The method of claim 1, further comprising scoring the at least a portion of the soft tissue with a scoring element.

8. The method of claim 7, wherein the score in the soft tissue is a partial thickness score.

9. The method of claim 1, further comprising cutting or cauterizing at least a portion of the soft tissue with an electrode.

10. The method of claim 9, wherein the cutting or the cauterizing comprises delivering radiofrequency energy to the soft tissue with an electrode coupled with the device.

11. The method of claim 9, wherein the cut in the soft tissue is a partial thickness cut.

12. The method of claim 1, further comprising cryogenically cooling at least a portion of the soft tissue.

13. A method for treating musculoskeletal tissue in a patient, said method comprising:
positioning a device having a working surface and a distal element adjacent the musculoskeletal tissue;
engaging the musculoskeletal tissue with the distal element;
stretching the tissue with the distal element into a stretched condition; and
cooling the working surface thereby chilling the stretched tissue to a cryogenic temperature so that the stretched tissue remains in the stretched condition following removal of the device.

14. The method of claim 13, wherein the positioning comprises positioning the device into a carpal tunnel of a hand.

15. The method of claim 13, wherein the tissue comprises a ligament.

16. The method of claim 13, wherein the tissue comprises plantar fascia or tissue adjacent thereto.

17. The method of claim 13, wherein the tissue is disposed in a joint.

18. The method of claim 13, wherein the distal element comprises an expandable member, and wherein the stretching comprises expanding the expandable member against the tissue.

19. The method of claim 18, wherein the expandable member comprises a balloon, and the expanding comprises inflating the balloon with a fluid.

20. The method of claim 19, wherein the stretching comprises expanding the balloon in a first direction while constraining the balloon from expanding in a second direction.

21. The method of claim 18, wherein the expandable member comprises two or more prongs and the stretching comprises pressing the prongs against the tissue.

22. The method of claim 18, wherein the expandable member comprises two or more prongs, and the stretching comprises engaging the tissue with the prongs and twisting the prongs.

23. The method of claim 13, wherein the distal element comprises a plurality of cooling tubes axially oriented and forming a generally cylindrical arrangement, and wherein the stretching comprises compressing the tubes so as to radially deflect at least a portion of the generally cylindrical arrangement outward against the tissue.

24. The method of claim 13, wherein the working surface comprises an outer surface of a balloon, and the cooling comprises inflating the balloon with a cryogenic fluid thereby reducing temperature of the working surface.

25. The method of claim 24, wherein the cooling comprises:
inflating the balloon with an inflation fluid delivered through a first lumen in the device; and
cooling the working surface of the balloon with the cryogenic fluid delivered through a second lumen in the device.

26. The method of claim 25, wherein the balloon comprises a first chamber in communication with the first lumen and a second chamber in communication with the second lumen.

27. The method of claim 13, wherein the working surface comprises an outer surface of one or more cooling tubes adjacent the distal element, and wherein the cooling comprises passing a cryogenic fluid through the one or more cooling tubes.

28. The method of claim 13, wherein the cooling comprises insulating at least a portion of the tissue from the cooling with an insulated portion of the device adjacent the working surface.

29. The method of claim 13, further comprising cutting the tissue with a cutting element prior to cooling the tissue.

30. The method of claim 29, wherein the cutting element comprises a scoring element, and the cutting comprises scoring the tissue.

31. The method of claim 29, wherein the cutting element comprises an electrode, and the cutting comprises delivering electrical energy to the tissue.

32. The method of claim 29, wherein the cut in the tissue is a partial thickness cut.

33. The method of claim 13, further comprising scoring the tissue.

34. The method of claim 33, wherein the score is a partial thickness score through the tissue.

35. The method of claim 13, further comprising cutting or cauterizing the tissue with an electrode on the device, the electrode delivering electrical energy to the tissue.

36. The method of claim 35, wherein the cut is a partial thickness cut through the tissue.

37. The method of claim 13, further comprising viewing the musculoskeletal tissue through an arthroscope.

38. A system for treating compressive neuropathy in a patient, said system comprising:
an elongate shaft having a proximal end, a distal end, and a lumen extending therebetween;
an expandable member adjacent the distal end of the shaft and having an expanded configuration and a collapsed configuration, the expandable member being fluidly coupled with the lumen, and wherein the expandable member is positionable into a space surrounding a nerve, and wherein in the expanded configuration the expandable member is configured to stretch tissue surrounding the nerve; and
a cryogenic fluid supplied through the lumen to the expandable member, wherein the cryogenic fluid expands the expandable member into the expanded configuration, and wherein the cryogenic fluid cools an outer surface of the expandable member thereby cooling the stretched tissue.

39. The system of claim 38, further comprising a shield adjacent the expandable member, the shield adapted to protect adjacent tissue from pressure exerted by the expandable member in the expanded configuration.

40. The system of claim 38, further comprising a scoring element disposed adjacent the expandable member, and adapted to score the stretched tissue.

41. The system of claim 38, further comprising an electrode disposed adjacent the expandable member, and adapted to cut or score the stretched tissue with electrical energy.

42. The system of claim 41, wherein the electrode is actuatable into an expanded configuration extending radially outward from the expandable member.

43. The system of claim 38, wherein the expandable member comprises a balloon.

44. A system for treating musculoskeletal tissue in a patient, said system comprising:
an elongate shaft having a proximal end, a distal end, and a lumen extending therebetween;
an expandable member adjacent the distal end of the shaft and having an expanded configuration and a collapsed configuration, wherein the expandable member is positionable into apposition with the musculoskeletal tissue, and wherein in the expanded configuration the expandable member is configured to stretch the tissue;
a cooling element adjacent the expandable member, and in fluid communication with the lumen; and
a cryogenic fluid supplied through the lumen to the cooling element so as to cryogenically cool the cooling element, and thereby cool the stretched tissue.

45. The system of claim 44, wherein the expandable member comprises a first balloon and the cooling element comprises a second balloon, the first and second balloons being concentrically disposed about the shaft.

46. The system of claim 44, wherein the first and the second balloons are expandable independently of one another.

47. The system of claim 44, further comprising an inner shaft having an inner shaft lumen and a port in the inner shaft in fluid communication therewith, the inner shaft disposed in the elongate shaft lumen thereby forming an annular space therebetween, wherein the cryogenic fluid circulates into and out of the cooling element via the annular space and the inner shaft port.

48. The system of claim 44, wherein the cooling element comprises a cooling tube disposed against the expandable member, the cooling tube formed into a plurality of peaks and valleys.

49. The system of claim 48, wherein the cooling tube is configured to expand with the expandable member.

50. The system of claim 48, wherein the cooling tube is coupled to the expandable member with a thread-like member.

51. The system of claim 44, wherein the cooling element comprises a plurality of cooling tubes interwoven to form a mesh, the mesh disposed over the expandable member.

52. The system of claim 44, further comprising a supporting element disposed adjacent the expandable member, wherein the supporting element prevents expansion of the expandable member in a first direction while allowing expansion in a second direction laterally away from the supporting element, thereby preventing the expandable member from expanding into tissue in the first direction.

53. The system of claim 44, further comprising an insulating member disposed adjacent the cooling element, the insulating member configured to protect tissue from being cooled by the cooling element.

54. The system of claim 44, further comprising an outer shaft having a central channel and a port in a sidewall of the outer shaft, wherein the expandable member is disposed in the central channel and is configured to expand in a first direction through the port while being constrained from expansion in a second direction by an opposite sidewall of the outer shaft.

55. The system of claim 44, wherein the expandable member comprises a plurality of cooling tubes extending axially along the elongate shaft and the cooling element comprises an outer surface of the cooling tubes, the cryogenic fluid flowing through the cooling tubes, and wherein axial compression of the cooling tubes expands the tubes radially outward into a cage-like structure.

56. The system of claim 44, wherein the expandable member comprises a plurality of actuatable prongs, the prongs configured to capture the tissue therebetween, and wherein actuation of the prongs stretches the tissue.

57. The system of claim 44, further comprising a scoring element disposed adjacent the expandable member, and adapted to score the tissue.

58. The system of claim 44, further comprising an electrode configured to deliver electrical energy to the tissue thereby cutting or scoring the tissue.
